(19) United States
(12) Patent Application Publication
(10) Pub. No.: US 2024/0021317 A1
(43) Pub. Date: Jan. 18, 2024

(54) Systems and Methods for Health Analysis

(71) Applicant: Brian Matthew Hood, Minnetrista, MN (US)

(72) Inventor: Brian Matthew Hood, Minnetrista, MN (US)

(21) Appl. No.: 17/812,547

(22) Filed: Jul. 14, 2022

(51) Int. Cl. G16H 50/30; A61B 5/00; A61B 5/16; A61B 5/11; G16H 10/60; G16H 40/20; G16H 50/20; G16H 70/00

(52) U.S. Cl. CPC G16H 50/30; A61B 5/746; A61B 5/165; A61B 5/1118; G16H 10/60; G16H 40/20; G16H 50/20; G16H 70/00

(57) ABSTRACT

A computing system includes a communication system configured to obtain sensor data indicative of performance, by an individual, of an activity indicative of a health status of the individual. The computing system further includes a health status system configured to generate a value indicative of the performance of the activity indicative of the health status of the individual based on the sensor data and compare the value of the performance of the activity indicative of the health status of the individual to a reference value of performance of the activity indicative of the health status of the individual. The computing system can further include an action signal genera tor configured to generate an action signal based on the comparison.

### SUMMARY

In one example, a computing system includes a communication system configured to obtain sensor data indicative of performance, by an individual, of an activity indicative of a health status of the individual. The computing system further includes a health status system configured to generate a value indicative of the performance of the activity indicative of the health status of the individual based on the sensor data and compare the value of the performance of the activity indicative of the health status of the individual to a reference value of performance of the activity indicative of the health status of the individual. The computing system can further include an action signal generator configured to generate an action signal based on the comparison.

In another example, a computer implemented method includes obtaining sensor data indicative of performance, by an individual, of an activity indicative of a health status of the individual and generating a value indicative of the performance of the activity indicative of the health status of the individual based on the sensor data. The method further includes comparing the value of the performance of the activity indicative of the health status of the individual to a reference value of performance of the activity indicative of the health status of the individual. The method can further include generating an action signal to provide an action based on the comparison.

In another example, a computing system architecture includes sensor configured to detect performance, by an individual, of an activity indicative of a health status of the individual and generate a sensor signal indicative of the detected performance and a computing system. The computing system, of the computing system architecture, and include a communication system configured to obtain the sensor signal and a health analysis system configured to generate a value indicative of the performance of the activity indicative of the health status of the individual based on the sensor signal and compare the value of the performance of the activity indicative of the health status of the individual to a reference value of performance of the activity indicative of the health status of the individual. The computing system, of the computing system architecture, can further include an action signal generator configured to generate an action signal based on the comparison.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

FIG. 1 is a block diagram of one example of a computing system architecture.

FIG. 2 is a flow diagram showing example operations of the health analysis system illustrated in FIG. 1.

FIGS. 3A-3C are pictorial illustrations showing example interface displays.

FIG. 4 is a block diagram showing one example of the architecture illustrated in FIG. 1 deployed in a remote server architecture.

FIG. 5-7 show examples of mobile devices that can be used in the architecture(s) shown in the previous figure(s).

FIG. 8 is a block diagram showing one example of a computing environment that can be used in the architecture(s) shown in the previous figure(s).

### DETAILED DESCRIPTION

Individuals with certain health issues (e.g., health conditions) and/or of a certain age can require additional care. For example, seniors may be candidates for moving into assisted living facilities (e.g., nursing homes, etc.) or other restricted living conditions, due to health issues or health risks, or both, for instance, a risk of falling, forgetfulness, medical conditions, etc. Often, these individuals desire to stay in their own homes, rather than moving into an assisted living facility or restricted living conditions. This desire can conflict with the advice of medical professionals and the wishes of caretakers, such as family, friends, etc., which can cause conflict. Additionally, should the individuals choose to stay in their own homes, there is an associated risk to their health given their health issues or an increased burden on their caretakers to monitor them, or both. Currently, monitoring the individuals can be ineffective for various reasons, for example, because of limited tracking of the individuals' conditions and/or activities. For example, some individuals may be provided a blood pressure sensor device which can monitor an individual's blood pressure and the resultant blood pressure data can be shared with a medical professional or with a caretaker. However, blood pressure is only a single factor, and thus, other factors that may indicate a health status of the individual may not be accounted for. Additionally, monitoring biomarkers, such as blood pressure, can be of limited usefulness without context into how the biomarker levels are affecting other aspects of their lives, or how other aspects of their lives are affecting the biomarker levels. In some examples, the monitoring relies solely, or mostly, on self-reporting, such that the individual must provide an accounting of the conditions or activities. However, for various reasons, the self-reporting may be inaccurate or incomplete, or both. For example, due to the individual's desire to stay in their home, the individual may inaccurately represent their conditions. In another example, the individual may not accurately recall events and thus may not accurately or fully report their conditions. Further, self-reporting may introduce human error into the measurement of the conditions and/or activities. Thus, current monitoring may be insufficient, incomplete, or inaccurate, or a combination thereof.

Activities of daily living (ADLs) can be used to measure changes in physical function of an individual for the purposes of health analysis and medical diagnosis. There are six ADLs: 1) walking independently; 2) feeding oneself; 3) dressing oneself; 4) bathing and grooming oneself; 5) controlling one's bladder and bowel; and 6) getting to and from the bathroom (e.g., toilet) oneself. The ability of the individual to perform ADLs can be used in health analysis and medical diagnosis.

Instrumental Activities of Daily Living (IADLs) can be used to measure a greater range of activities needed for independence and to spot disabilities which may not be identified when utilizing ADLs alone. IADLs are more complex activities that require more organization and complex thinking skills. There are eight IADLs: 1) using the telephone; 2) going shopping; 3) preparing food; 4) performing housekeeping; 5) doing laundry; 6) managing personal travel; 7) taking medication without supervision; and 8) managing one's own finances. The ability of the individual to perform IADLs can be used in health analysis and medical diagnosis.

ADL performance tends to be more sensitive to physical decline (e.g., decline in mobility), whereas IADL performance tends to be more sensitive to cognitive decline. While ADLs and IADLs can be used in health analysis and medical diagnosis, current tracking of performance of these activities can be limited. For example, current tracking may be reliant on self-reporting, which, as described above, can be insufficient, inaccurate, and/or incomplete. In another example, a device, such as an at-home blood pressure monitor, may only provide data for one factor and may not provide comprehensive data for all or any of the ADLs or IADLs, or both. Additionally, other forms of monitoring such as home visits, in-home medical assistance, doctor visits, etc. may be infrequent or otherwise only periodic.

Further, while ADLs and IADLs are useful, there are additional activities that can be useful in health analysis and medical diagnosis which are not accounted for by ADLs and IADLs. These additional activities are herein referred to as mental health activities (MHAs).

MHAs contribute to maintaining health over the long term and can be used in health analysis and medical diagnosis. MHAs, as discussed herein, fall into three categories: 1) social activities (e.g., bridge club, walking each day, weekly coffee with a friend, etc.); 2) spiritual activities (e.g., attending spiritual service, such as church service, going to spiritual study, such as bible study, attending weekly spiritual group with a specific focus area, such as weekly church group with a specific focus area, etc.); 3) volunteer work (e.g., visiting people in nursing home, serving meals at a soup kitchen, serving communion to those who cannot attend church, etc.). While example activities of each category are given, it is to be understood that various other activities belonging to those categories are also contemplated herein. As will be described in greater detail below, the MHAs can be customizable by the individual, or other users, to provide individual-specific monitoring. MHA monitoring can be an important factor that is not considered in monitoring other activities, such as ADLs and/or IADLs. MHAs can be an indicator of the health status of an individual. Additionally, in some examples, an individual may be performing ADLs and IADLs, but are not performing MHAs, and thus their health status could be deteriorating but such deterioration would not be indicated by merely monitoring performance of ADLs and IADLs.

Described herein is a computing system architecture that can provide accurate monitoring of ADLs, IADLs, and MHAs for purposes of health analysis and medical diagnosis. The computing system architecture can utilize a variety of sensor devices disposed within an individual's home and/or personal items (e.g., mobile devices, vehicles, appliances etc.) as well as wearable sensor devices for detection of activity performance. The detected activities performance can be compared to reference activities performance, such as desired (e.g., goal) activities performance, historical activities performance, and/or standard activities performance, to identify deviation. The activities and activities performance can be customized and set by the individual, caretakers, healthcare workers, etc. Thus, the computing system architecture provides customizable tracking and analysis based on customizable activities and customizable reference activities performance. Based on the detected performances or identified deviations, or both, various action signals can be generated by the computing system architecture. The described computing system architecture can enable individuals to stay in their own homes longer, provide more accurate, more complete, and more holistic measurement of activities for purposes of health analysis and medical diagnosis, reduce conflict with and/or worry of caretakers (e.g., family, friends, etc.). Additionally, the computing system architecture can provide real time indications (e.g., alerts, notifications, displays, etc.). The displays can illustrate detected performance of activities, trends, comparisons to reference levels of performance of activities, as well as biomarker data. In addition, the computing system architecture can generate various action signals to coordinate care of the individual, such as scheduling appointments, arranging transportation, contacting emergency services, providing reminder calls, such as reminder calls to take medication, as well as various other actions. The detected levels of activity performance and biomarkers can be displayed to designated users (which can be selectively customized by the individual) such as caretaker(s) (e.g., friends, family, etc.), healthcare workers (e.g., doctors, nurses, etc.), as well as healthcare provider systems (e.g., hospital system, clinic system, etc.) such that the other users and systems can be presented with an indication of the individual's daily activity and biomarkers as well an indication of the individual's activities and biomarkers over time. Additionally, the computing system architecture provides users with remote tracking and analysis of individuals, such that distances can be maintained between the user(s) and the individual, for example, when distance must be maintained between the user(s) and the individuals.

Additionally, the described computing system architecture can provide proactive identification of potential health issues so that action can be taken before a larger problem develops. Early detection of certain conditions (e.g., dementia) can allow treatments to treat the condition, delay onset, and/or increase quality of life.

It will be noted that while particular examples described herein are discussed with reference to seniors (e.g., elderly individuals) the systems and methods herein are applicable for individuals of all ages and/or having any various health issues.

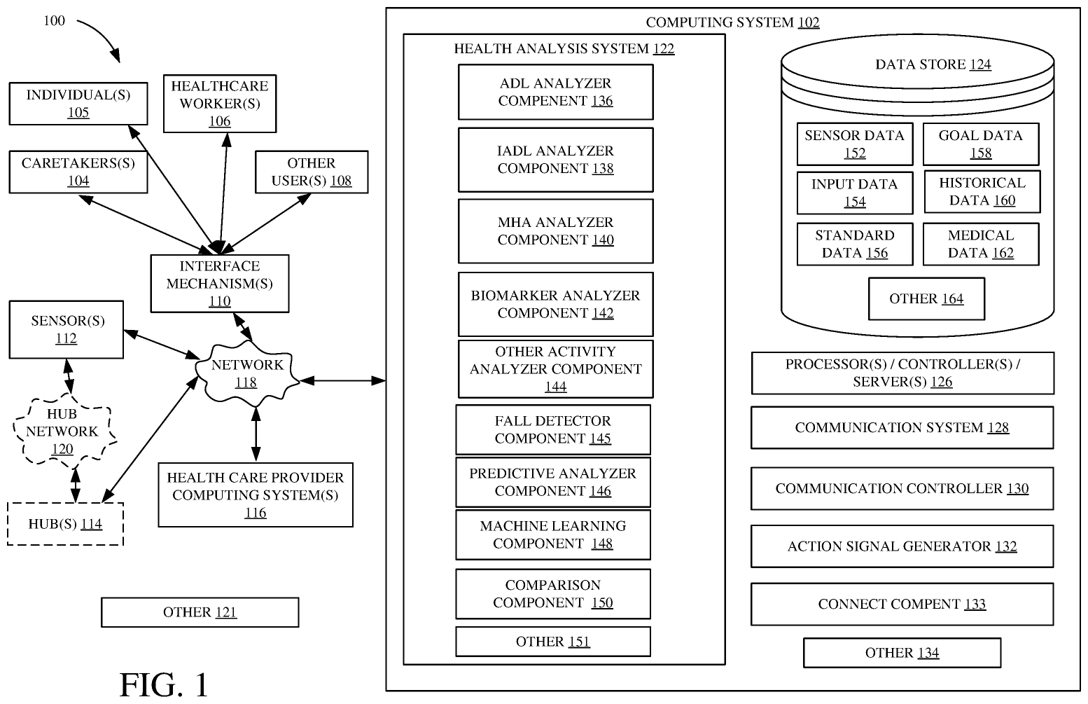

FIG. 1 is a block diagram of one example of a computing system architecture 100 having, among other things, a computing system 102 including a health analysis system 122. FIG. 1 shows that architecture 100 can include computing system 102, one or more users, such as one or more caretakers 104, one or more individual(s) 105, one or more healthcare workers 106, as well as various other users 108. Architecture 100 can further include one or more interface mechanisms 110, one or more sensors 112, one or more hub devices 114, one or more health care provider computing systems 116, network 118, hub network 120, and can include various other items 121 as well. Computing system 102, itself, can include health analysis system 122, data store 124, one or more processors, controllers, or servers 126, communication system 128, communication controller 130, action signal generator 132, connect component 133, and can include various other items 134 as well. Health analysis system 122, itself, can include ADL analyzer component 136, IADL analyzer component 138, MHA analyzer component 140, biomarker analyzer component 142, other activity analyzer component 144, fall detector component 145, predictive analyzer component 146, machine learning component 148, comparison component 150, and can include various other items 151 as well. Data store 124, itself, can include sensor data 152, input data 154, standard data 156, goal data 158, historical data 160, medical data 162, and can include various other data 164 as well. It will be understood that us used herein, individual (e.g., individuals 105), refer to persons whose health status is being monitored (e.g., monitoring activities, such as ADLs, IADLs, MHAs and biomarkers, etc.), such as seniors or other people with health conditions.

Computing system 102 is configured to interact with other components and systems of computing architecture 100. For instance, communication controller 130 is configured to control communication system 128. Communication system 128 is used to communicate between components of computing system 102 or with other items of architecture 100 over network 118. Network 118 can be any of a wide variety of different types of networks such as the Internet, a cellular network, a wide area network (WAN), a local area network (LAN), a controller area network (CAN), a near-field communication network, or any of a wide variety of other networks or combinations of networks or communication systems. Additionally, computing system 102 includes a connect component 133 which provides customizable connectivity and information sharing with select devices based on user input. Thus, in one example, a user can use connect component 133, via communication system 128, to add or delete certain devices (e.g., sensors 112, user devices, such as interface mechanisms 110, remote computing systems, such as health care provider computing systems 116) with which computing system 102 is to interact with.

Various remote users, such as caretakers 104, individuals 105, healthcare workers 106, as well as other users 108, are shown interacting with computing system 102, such as through user interface mechanisms 110 and over network 118. Interface mechanisms 110 can be a wide variety of different types of systems. For example, interface mechanisms 110 can be a computing system, such as a mobile device. Additionally, other computing systems and devices are shown interacting with computing system 102 over network 118, such as medical provider computing systems 116, sensors 112 and hubs 114. Health care provider computing system 116 can be, in one example, a computing system of health care provider, such as the computing system of a health professional or health facility organization (e.g., clinic(s), hospital(s), etc.). It will be noted that in some examples, sensors 112 interact with hubs 114 over a hub network 120 (e.g., a hub area network such as a LAN). In some examples, one or more of sensors 112 may communicate only with hubs 114 over a hub network 120 and thus, information from sensors 112 is communicated to computing system 102 via hubs 114 over network 118. In some examples, one or more sensors 112 may communicate with computing system 102 over network 118 or with hubs 114 over hub network 120. The users, such as caretakers 104, healthcare workers 106, and other users 108 can be designated as allowed users by the individual 105 such that those designated users have access to or are provided with select or all of the information provided by computing system 102. Additionally, the systems, such as health care provider computing system 116, can be designated as allowed systems by the individual 105 such that those designated system have access to or are provided with select or all of the information provided by computing system 102.

Sensors 112 can include various types of sensors, including various sensors placed in an individual's home as well as in items of the individual (e.g., smart devices). Sensors 112 are configured to provide sensor data indicative of activity of the individual as well as conditions of the individual. For instance, sensors 112 provide sensor data indicative of an individual's performance of ADLs, IADLs, MHAs, as well as sensor data indicative of an individual's biomarkers (e.g., blood pressure, heart rate, sleep markers, etc.). Sensors can include motion sensors to detect an individual's presence and motion in a given location, contact sensors, such as contact sensors configured to detect the opening of doors or windows, sensors, such as 4D imaging sensors, that detect when someone is lying on the ground, standing, or sitting (e.g., in a chair), environmental sensors, such as humidity sensors, temperature sensors, etc. In some examples, sensors 112 are a part of other items used or owned by the individual. For example, many items in a household may be “smart devices” and thus may include a variety of sensors that communicate various information, for instance items owned by the individual (e.g., fridge, stove, microwave, washer, dryer, television, thermostat, smoke/carbon monoxide detectors, etc.) may include various sensors that provide information relative to the status and operations of the appliances, as well as information relative to the environment of the device (e.g., temperature, humidity, air conditions, etc.). Additionally, some individuals may operate or carry smart devices (e.g., mobile device, smart watches, vehicles, etc.) that include various sensors that provide information relative to the status and operations of the devices, as well as information relative to the environment of the devices. Additionally, some of these sensors 112 may provide position and/or location information, such as global positioning information. For instance, an individual's mobile device (e.g., smart phone), a smart watch, and vehicle may provide position information that indicates a location of the device and thus may be used to indicate a location of the individual. Additionally, furniture used by the individual may include or be provided with a sensor that indicates use of the furniture. For example, a smart sleep pad may detect the presence of the user in a bed as well as provide various sleep biomarker data, such as sleep cycle information, snoring and breathing disturbance information, sleeping heart rate, as well as various other information. Additionally, sensors 112 can include biomarker sensor devices, such as heart rate monitors, blood pressure monitors, blood glucose monitors, EKG monitors, pulse oximeters, smart weight scales, wearable smart patches, ultrasound devices, as well as various other biomarker sensors. These are only some examples of sensors 112, it will be understood that various other sensors are also contemplated herein. Additionally, it will be understood that various imaging devices (e.g., cameras) are also contemplated herein, however, in some examples, the systems and methods disclosed herein can be achieved without the use of imaging devices. For example, some individuals may prefer not to have imaging devices placed in their homes or items. Additionally, in some examples, sensors 112 can include implantable sensor technology.

Before discussing the overall operation of computing system 102, a brief description of some of the items in computing system 102, and their operation, will first be provided.

Communication system 128 can include wireless communication logic, which can be substantially any wireless communication system that can be used by the systems and components of computing system 102 to communicate information to other items. In some examples, communication system 128 communicates over a BUS (e.g., address bus, data bus, control bus, etc.) In another example, communication system communicates over a network to communicate information between those items. This information can include user inputs provided by various remote users, the various data in data store 124, sensor data provided by sensors 112, information and/or controls output by processors/controllers/servers 126, as well as outputs provided by health analysis system 122, as well as various other information. Thus, in some examples, communication system 128 can be a wireless communication system, a wired communication system, or include a combination of both.

Sensor data 152 includes data provided by sensors 112, and is indicative of activity of the individual 105 as well as conditions of the individual. For instance, sensor data 152 can be indicative of performance, by an individual 105, of ADLs, IADLs, MHAs, as well as performance, by an individual 105, of various other activities. Sensor data 152 can also be indicative of various biomarkers of individual 105. Input data 154 includes data provided by user input, which can include a variety of information, such as self-reported activity data, self-reported biomarker data, self-reported symptom data, as well as various other information. Standard data 156 can include data relative to a standard or typical individual, such as standard or typical activity data, standard or typical biomarker data, standard or typical symptom data, as well as various other data.

Goal data 158 includes data indicative of goal (e.g., desired) levels (e.g., values, metrics, performance, etc.) of activities (i.e., ADLs, IADLs, MHAs, etc.) and/or biomarkers. For instance, an individual 105 can set goal levels of their ADLs (e.g., go to bathroom 6 times per day), IADLs (e.g., take medication by 9:00 AM), MHAs (e.g., go to church once a week), or biomarkers (e.g., blood pressure of 125/85). In this way, an individual's actual levels can be tracked (e.g., based on sensor data from sensors 112, inputs provided by the users, etc.) and compared to goal levels. In other examples, other users, such as caretakers 104 or healthcare workers 106, can, alternatively or additionally to the individual 105, set one or more goal levels of an individual's activities and/or biomarkers.

Historical data 160 includes data indicative of historical levels of an individual's activities and/or biomarkers. The historical levels can be filtered (e.g., by activity or biomarker, by time span, etc.), aggregated (e.g., mean, median, other statistical distribution), or otherwise manipulated. Historical data 160 can also include baseline health levels (e.g., baseline ADL levels, baseline IADL levels, baseline MHA levels, baseline biomarker levels, etc.). For example, health analysis system 122 can determine a baseline for each individual ADL, IADL, MHA, and/or biomarker (as well as determine a baseline overall for ADL, IADL, MHA, and/or biomarkers). In some examples, health analysis system can develop a baseline based on levels over a given period of time (e.g., 30 days, one week, etc.). In developing a baseline, outliers can be optionally removed. The values detected during the baseline period can be aggregated in various ways (e.g., averaged, etc.). Additionally, the health analysis system 122 can redetermine (e.g., update, refresh, etc.) the baseline with more current data automatically (e.g., based upon a trigger such as a period of time, a new condition, an event, etc.) and/or based upon a user input.

Medical data 162 can includes data indicative of medical information of an individual 105, such as medical and health conditions, predispositions (e.g., genetic predispositions, family history, other predispositions, etc.), as well as various other medical information. Medical data 162 can be provided by one or more of the users (e.g., 104, 105, 106, etc.) as well as the health care provider of the individual (e.g., 116). Data store 124 can include various other data 164.

Health analysis system 122 obtains various data and other inputs. Based on the various data and other inputs, health analysis system 122 provides various outputs on the basis of which various action signals can be generated via action signal generator 132. For example, health analysis system can determine an individual's current health status (e.g., ADL levels, IADL levels, MHA levels, biomarker levels, overall levels, deviations, trends, etc.) and can generate a variety of action signals based upon this determination, such as an action signal to provide alerts or notification to users, action signals to control interface mechanisms to provide indications (e.g., displays) of the determination, as well as action signals to coordinate services, such as emergency services (e.g., ambulance), scheduling an appointment, arranging transportation (e.g., medical transportation, contract transportation, such as taxi or other ride services, etc.), etc. Additionally, health analysis system 122 can determine an individual's current health status, as well as determine that an individual's current health status deviates from a reference (e.g., goal, historical, standard, etc.) health status and can generate various action signal based upon this determination.

ADL analyzer component 136 can include various logic or memory storing instructions that are executed by the one or more processors/controllers/servers 126 to provide tracking and analysis of an individual's performance of ADLs, on the basis of the various data and other inputs obtained by health analysis system 122. ADL analyzer component 136 can generate outputs indicative of an individual's ADL levels (e.g., values, metrics, performance, etc.). For example, ADL analyzer component 136 can generate outputs indicative of levels of individual ADL activities (e.g., how many times individual bathed in a given period) as well as an overall ADL level (e.g., mobility score). The overall level can be an aggregation of individual levels of different ADLs.

IADL analyzer component 138 can include various logic or memory storing instructions that are executed by the one or more processors/controllers/servers 126 to provide tracking and analysis of an individual's performance of IADLs, on the basis of the various data and other inputs obtained by health analysis system 122. IADL analyzer component 138 can generate outputs indicative of an individual's IADL levels (e.g., values, metrics, performance, etc.). For example, IADL analyzer component 138 can generate outputs indicative of levels of individual IADL activities (e.g., how many times an individual left stove on in a given period) as well as an overall IADL level (e.g., cognitive score). The overall level can be an aggregation of individual levels of different IADLs.

MHA analyzer component 140 can include various logic or memory storing instructions that are executed by the one or more processors/controllers/servers 126 to provide tracking and analysis of an individual's performance of MHAs, on the basis of the various data and other inputs obtained by health analysis system 122. MHA analyzer component 140 can generate outputs indicative of an individual's MHA levels (e.g., values, metrics, performance, etc.). For example, MHA analyzer component 140 can generate outputs indicative of levels of individual MHA activities (e.g., how many times an individual went to coffee with friends in a given period) as well as an overall MHA level (e.g., mental health score). The overall level can be an aggregation of individual levels of different MHAs.

Biomarker analyzer component 142 can include various logic or memory storing instructions that are executed by the one or more processors/controllers/servers 126 to provide tracking and analysis of an individual's biomarkers, on the basis of the various data and other inputs obtained by health analysis system 122. Biomarker analyzer component 142 can generate outputs indicative of an individual's biomarker levels (e.g., values, metrics, performance, etc.). For example, biomarker analyzer component 142 can generate outputs indicative of levels of individual biomarkers (e.g., blood pressure, pulse, etc.) as well as an overall biomarker level (e.g., biomarker score). The overall level can be an aggregation of individual levels of different biomarkers.

Other activity analyzer component 144 can include various logic or memory storing instructions that are executed by the one or more processors/controllers/servers 126 to provide tracking and analysis of an individual's various other activities, on the basis of the various data and other inputs obtained by health analysis system 122. Other activity analyzer component 144 can generate outputs indicative of an individual's other activity levels (e.g., values, metrics, performance, etc.). For example, other activity analyzer component 144 can generate outputs indicative of levels of individual other activities as well as an overall other activity level. The overall level can be an aggregation of individual levels of the different other activities.

Fall detector component 145 can include various logic or memory storing instructions that are executed by the one or more processors/controllers/servers 126 to provided fall detection on the basis of various data obtained by health analysis system and/or based on the various outputs provide by other items of health analysis system 122. Fall detector component 145 can detect when (e.g., what time of day, what date, etc.) and where (e.g., what room of the house, etc.) an individual falls, track how many falls occur over given periods of time, and provide outputs indicative of fall occurrences as well as fall levels. Additionally, fall detector component 145 can determine if an individual was able to get back up after a detected fall or not.

Predictive analyzer component 146 can include various logic or memory storing instructions that are executed by the one or more processors/controllers/servers 126 to provide predictive health analysis on the basis of the various data obtained by health analysis system 122 and/or based on the various outputs provided by other items of health analysis system 122. Predictive analyzer component 146 can predict that an individual has or will have a one or more health conditions. For example, based upon data (e.g., sensor data, such as motion sensor data, location data, or both, etc.) that indicates an individual is home but has not moved for a given period of time and/or during a certain time of the day, predictive analyzer component 146 may provide an output indicating that the individual is incapacitated, such as has suffered a fall. This is merely an example. In some examples, predictive analyzer component 146 can determine if the individual has a potential injury. For example, if a fall is detected by fall detector component 145 and the mobility score and/or mental health score decreases subsequent to the detected fall, then predictive analyzer component can generate an output indicative of a likely injury.

Machine learning component 148 can include various logic or memory storing instructions that are executed by the one or more processors/controllers/servers 126 to provide machine learning. Machine learning component 148 can improve the determinations made by health analysis system 122 by improving the algorithmic process for the determination, such as by improving the recognition of activities and conditions of the individual that indicate current or potential health conditions. For example, machine learning logic 410 can learn relationships between activities, characteristics, factors, or conditions that affect the health status of the individual. Machine learning component 148 can also utilize a closed-loop style learning algorithm such as one or more forms of supervised machine learning.

Comparison component 150 can compare detected levels (e.g., ADL levels, IADL levels, MHA levels, biomarker levels) to reference levels (e.g., goal levels, historical levels, standard levels, etc.) to determine deviation. Additionally, in some examples, the deviation between detected levels and reference levels can be compared to a threshold, for instance, a threshold level of deviation. Comparison component 150 can provide an output indicative of these comparisons. As an illustrative example, comparison component 150 can determine that detected levels deviate from goal levels to a certain degree, however the detected levels may not deviate from historical/baseline levels, or only deviate from historical/baseline levels to a lesser degree, and thus, the deviation may not be a cause for concern, or may lead to a different determination than just considering deviation from goal levels on their own.

Based on the various outputs provided by items of health analysis system 122, as well as based on the data of data store 124, health analysis system 122 can generate a variety of action signals, via action signal generator 132, to provide a variety of actions, for instance to provide various indications (e.g., display, alert, notification, etc.) on an interface mechanism 110, to provide recommendations (e.g., recommend to see a doctor, schedule a certain service, etc.), to contact emergency services (e.g., request an ambulance), to interact with a health care provider, such as system 116, to provide indications or to schedule services (e.g., medical appointments), contact the individual, such as to provide reminders to perform certain activities (e.g., take medication, eat, go to church, bathe, etc.), etc. These are merely some examples of the actions that can be provided.

In one example, based on an output indicative of a detected fall, from fall detector component 145, health analysis system 122 can generate an action signal to provide an alert or other indication indicative of the fall data, such as to other users (e.g., 104, 106, 108), healthcare provider systems (e.g., 116), as well as contact emergency services (e.g., ambulance). In one example, based on an output indicative of a detected fall, from fall detector component 145, health analysis system 122 can use a voice prompt, such as through an individual's device (e.g., interface mechanism 110, hubs 114, etc.) to inquire if the individual requires assistance. In other example, computing system 102 can receive voice commands from the individual (or another user), such as a voice command to provide assistance (e.g., “help”, “call ambulance”, “call John/Jane”, etc.). In some examples, based on the various outputs provided by items of health analysis system 122, and/or based on data from data store 124, health analysis system 122 can generate a control signal to automated unlock of one or more doors of the individual's home, for example, upon detection or determination of an emergency event. For example, upon detection of a fall by fall detector component 145, health analysis system 122 can generate a control signal to unlock one or more doors of the individual's home (e.g., front door, back door, etc.). In this way, emergency personnel will have easier access to the individual's home and will not be required to break a door or window in the event of an emergency. In some examples, the individual 105 or other users (e.g., 104, 106, and/or 108) can customize which events are to be considered emergency events for purposes of automated control. In one example, based on an output indicative of a likely injury (such as a likely injury suffered after a detected fall) from predictive analyzer component 146, health analysis system 122 can generate a control signal to provide an alert or other indication to other users (e.g., 104, 106, 108, etc.) or a health care provider system (e.g., 116) In another example, based on an output indicative of a likely injury from predictive analyzer component 146, health analysis system 122 can generate a control signal to automatically schedule medical services with a health care provider (e.g., 116) and/or a specific health care worker (e.g., 106) such as by automatically scheduling an appointment. These are just some examples.

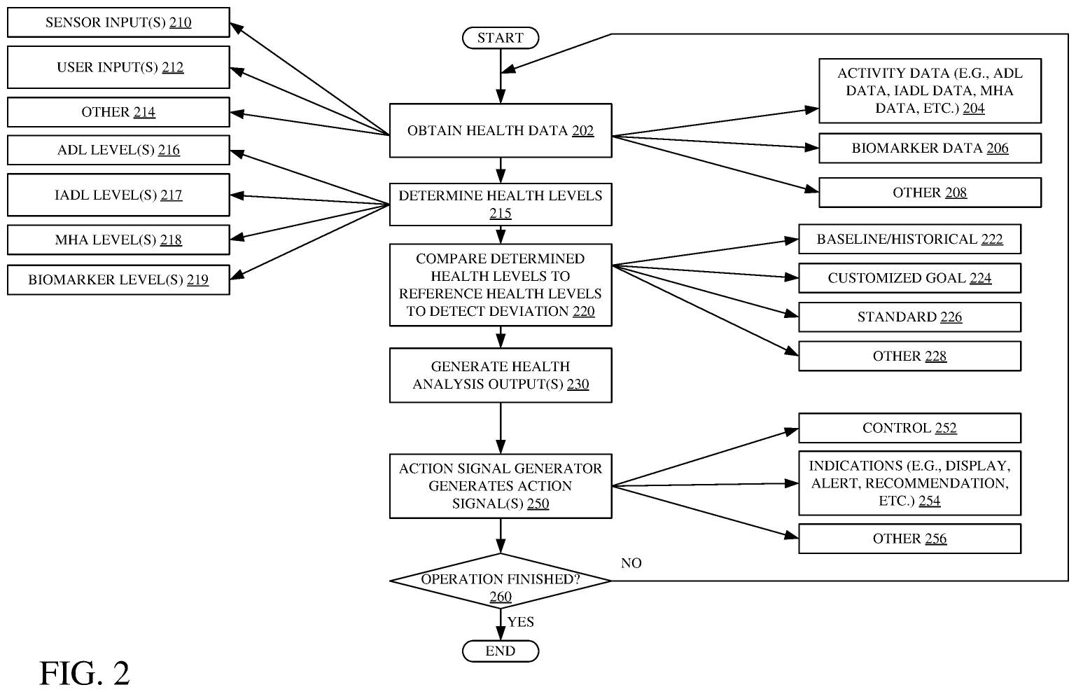

FIG. 2 is a flow diagram showing an example of the operation of the health analysis system 122 shown in FIG. 1 in determining a health status of an individual. While the operation will be described in accordance with computing system 102, it is to be understood that other computing systems with a health analysis system 122 can be used as well.

Processing begins at block 202 where health analysis system 122 obtains various data indicative of an individual's health status. The data may include various activity data (e.g., ADL data, IADL data, MHA data, other activity data, etc.) as indicated by block 204. The data may include biomarker data as indicated by block 206. The data may include various other data as indicated by block 208. The data may be obtained from sensors, such as sensors 112, as indicated by block 210. The data may be obtained from user inputs, such as one or more inputs from one or more remote users (e.g., caretakers 104, individuals 105, healthcare workers 106, other users 108) as indicated by block 212. The data may be obtained from various other sources, such as health care provider systems (e.g., 116), etc.

Once the data has been obtained at block 202, processing proceeds at block 215 where health analysis system 122 determines various health levels of the individual. For example, ADL analyzer component 136 can determine and output ADL level(s), as indicated by block 216. IADL analyzer component 138 can determine and output IADL level(s), as indicated by block 217. MHA analyzer component 140 can determine and output MHA level(s), as indicated by block 218. Biomarker analyzer component 142 can determine and output biomarker level(s), as indicated by block 219. Additionally, it will be noted that various other outputs can be generated. For example, other activity analyzer component 144 can determine and output other activity level(s).

Once the health levels are determined at block 215, processing proceeds at 220 where health analysis system 122 compares the determined health levels to reference health levels. For example, comparison component 150 can compare one or more determined health levels (e.g., ADL level(s), IADL level(s), MHA level(s), biomarker level(s), other level(s)) to reference health levels. The reference health levels can be based upon historical and/or baseline health levels, as indicated by block 222. As indicated by block 224, the reference health levels can be based upon customizable health level goals provided by a user, such as one or more of an individual (e.g., 105), a caretaker (e.g., 104), a healthcare worker (e.g., 106), as well as various other users. The reference health levels can be based upon standard health levels, as indicated by block 226. For example, standard health levels for similar individuals. The reference health levels can be based upon various other criteria, as indicated by block 228. In some examples, the reference health levels can be based upon a combination of the above criteria or different criteria can be used for the different health indicators (e.g., ADLs, IADLs, MHAs, biomarkers, etc.).

Once the health levels have been determined at block 215 and any desired comparisons have been implemented at block 220, processing proceeds at block 230 where health analysis system 122 outputs the determined health levels and comparison results. Processing proceeds to block 250 where health analysis system 122 generates one or more action signals, via action signal generator 132, based upon the one or more determined health levels and/or comparison results. For instance, action signals can be generated to control one or more items of architecture 100, for instance, communication controller 130 can control communication system 128 to contact emergency services (e.g., ambulance) or to schedule health care services (e.g., doctor appointments, etc.) with a health care provider or health care worker, for instance by interacting with a health care provider computing system (e.g., 116) or a health care worker (e.g., 106). Additionally, or alternatively, action signals can be generated to provide various displays or other indications (e.g., alerts, notifications, etc.), based upon the one or more determined health levels and/or comparison results. For example, a display or other indication (e.g., alert, notification, etc.) can be provided to a remote user (e.g., 104, 105, 106, 108) on an interface mechanism (e.g., 110) as well as on an interface corresponding to a remote computing system, such as health care provider computing system 116. Various other action signals can also be generated, as indicated by block 256.

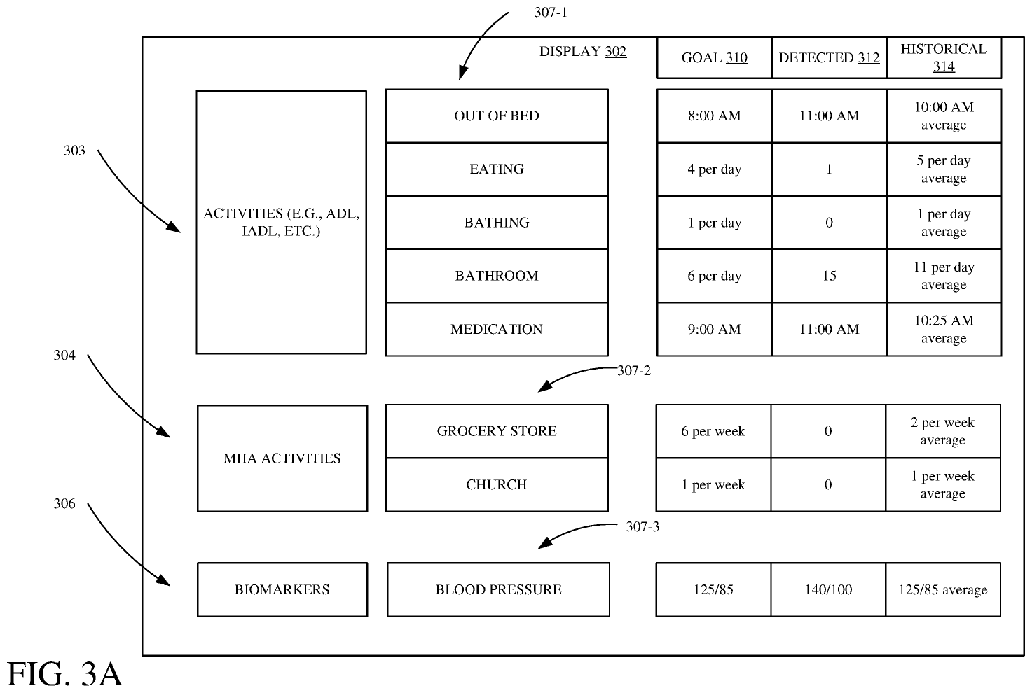

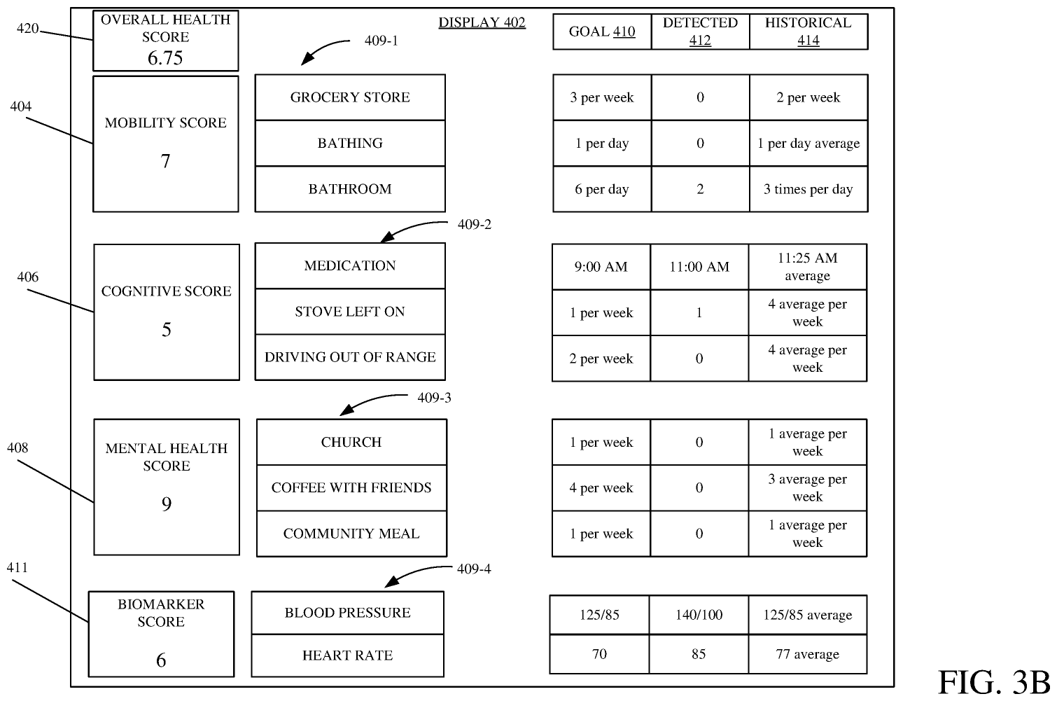

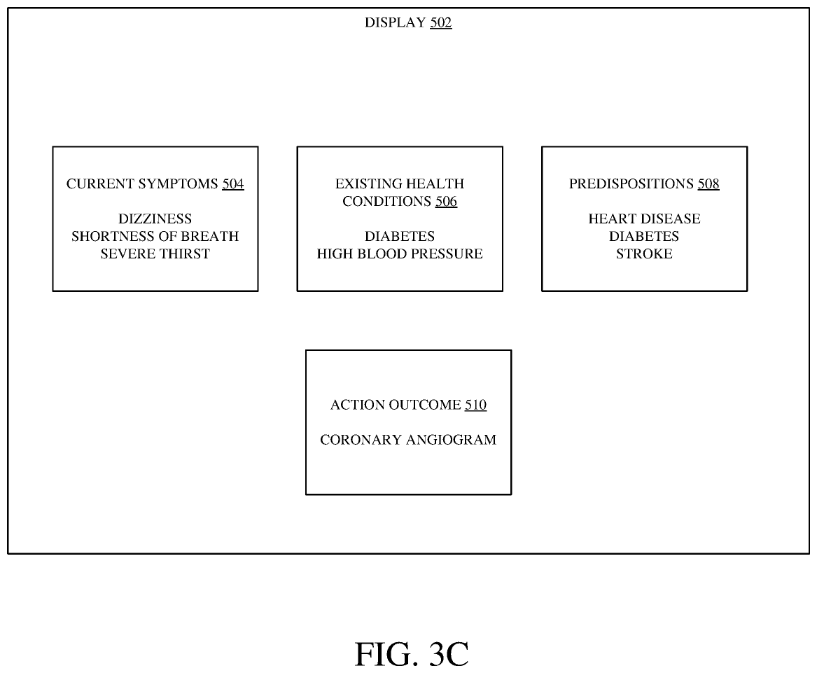

FIG. 3A is a pictorial illustration showing one example display 302 that can be provided by health analysis system 122. As illustrated in FIG. 3A, display 302 can include activities rows 303, MHAs row 304, and biomarkers row 306. Each row can have corresponding and customizable tracked health status indicators 307. For instance, activities row 302 includes tracked health status indicators “OUT OF BED”, “EATING”, “BATHING”, “BATHROOM”, and “MEDICATION” indicated generally at 307-1, MHAs row 304 includes tracked health status indicators “GROCERY STORE” and “CHURCH” indicated generally at 307-2, and biomarkers row 306 includes tracked health status indicator “BLOOD PRESSURE” indicated generally at 307-3. It will be understood that these are examples only and that in other examples more or less health status indicators can be utilized.

Display 302 also includes goal column 310 which displays corresponding goal values for the corresponding tracked health status indicators 307. For example, for the tracked indicator “OUT OF BED” there is a corresponding reference value of 8:00 AM indicating a goal to get out of bed by 8:00 AM on a daily basis. Display 302 also includes detected column 312 which displays corresponding detected values for the corresponding tracked indicators. The detected values can be displayed on a per time basis based on the reference value, for example, the detected value corresponding to the tracked indicator “BATHROOM” is a value indicative of how many trips to the bathroom the individual has made that day, whereas the detected value corresponding to the tracked indicator “GROCERY STORE” is a value indicative of how many trips to the grocery store the individual has made that week. Additionally, it should be understood that the detected values can be generated by health analysis system 122 based on sensor data provided by sensors 112.

Display 302 also includes historical column 312 which displays corresponding historical values (e.g., baseline values, etc.) for the corresponding tracked indicators. For example, for the tracked indicator “MEDICATION” there is a corresponding historical value of 10:25 AM average indicating that the individual has, on average, taken their medication at 10:25 AM over a given period of time (e.g., weekly, monthly, etc.).

It will be appreciated that the various display elements on display 302 can be altered or displayed in different ways, for example, one or more of the values can be distinguished from one or more other values on the display, such as by altering the appearance (e.g., font, size, color, etc.).

FIG. 3B is a pictorial illustration showing one example display 402 that can be provided by health analysis system 122. As illustrated, display 402 can include mobility score 404, cognitive score 406, mental health score 408, and biomarker score 411. In the example illustrated, each score is based on different tracked health status indicators 409. In the example illustrated, mobility score 404 is based upon values of corresponding tracked ADLs. In the example of FIG. 3B, the corresponding tracked ADLs “GROCERY STORE”, “BATHING”, and “BATHROOM” are indicated generally at 409-1. In the illustrated example, cognitive score 406 is based upon values of corresponding tracked IADLs. In the example of FIG. 3B, the tracked IADLs “MEDICATION”, “STOVE LEFT ON”, and “DRIVING OUT OF RANGE” are indicated generally at 409-2. In the example illustrated, mental health score 408 is based upon values of corresponding tracked MHAs. In the example of FIG. 3B, the tracked MHAs “CHURCH”, “COFEE WITH FRIENDS”, and “COMMUNITY MEAL” are indicated generally at 409-3. In the illustrated example, biomarker score 411 is based upon values of corresponding tracked biomarkers. In the example of FIG. 3B, the tracked biomarkers “BLOOD PRESSURE” and “HEART RATE” are indicated generally at 409-4.

Display 402 also includes goal column 410 which displays corresponding goal values for the corresponding tracked health status indicators. For example, for the tracked indicator “STOVE LEFT ON” there is a corresponding reference value of once per week indicating a goal to only leave the stove on once a week.

Display 402 also includes detected column 412 which displays corresponding detected values for the corresponding tracked indicators. The detected values can be displayed on a per time basis based on the reference value, for example, the detected value corresponding to the tracked indicator “BATHROOM” is a value indicative of how many trips to the bathroom the individual has made that day, whereas the detected value corresponding to the tracked indicator “COFFEE WITH FRIENDS” is a value indicative of how many trips to meet friends for coffee the individual has made that week. Additionally, it should be understood that the detected values can be generated by health analysis system 122 based on sensor data provided by sensors 112.

Display 402 includes historical column 412 which displays corresponding historical values (e.g., baseline values, etc.) for the corresponding tracked indicators. For example, for the tracked indicator “DRIVING OUT OF RANGE” there is a corresponding historical value of 4 average per week indicating that the individual has, on average, driven out of range 4 times per week over a given time period (e.g., weekly, monthly, etc.).

Display 402 can also include an overall health score 420. Overall health score 420 is based on the mobility score 404, cognitive score 406, mental health score 408, and biomarker score 411. The value for overall health score 420 can be the result of an aggregation of the values corresponding to the mobility score 404, the cognitive score 406, the mental health score 408, and the biomarker score 411, for instance, in the illustrated example, overall health score 420 is an average of the corresponding mobility score 404, cognitive score 406, mental health score 408, and biomarker score 411. This is just one example.

It will be appreciated that the various display elements on display 402 can be altered or displayed in different ways, for example, one or more of the values can be distinguished from one or more other values on the display, such as by altering the appearance (e.g., font, size, color, etc.).

The score values (e.g., 404, 406, 408) can be determined and output by health analysis system 122 based on goal levels, detected health levels, and historical/baseline health levels. In some examples, the scores are based on the deviation of the detected health levels from either or both of the reference levels (e.g., goal levels) and the historical/baseline levels. For instance, deviation of the detected level from the goal level may be mitigated (e.g., reduced in weight) where the detected level is nearer to or consistent with the historical/baseline level. Additionally, the score values can be determined and output by health analysis system 122 based on various criteria and weighting, which can be set by user(s), pre-stored, and/or learned. For example, deviation in leaving the stove on may be weighted more heavily than driving out of range for cognitive scoring. In another example, increased trips to the bathroom may be mitigated (e.g., reduced in weight) where the individual has also eaten more. Additionally, weights of tracked indicators can be based on conditions of the individual, such as the individual's particular medical condition, for instance leaving the stove on may be weighted more heavily for an individual with preexisting cognitive conditions and/or predispositions for cognitive conditions as compared to an individual with a heart condition.

Also, it will be noted that while certain tracked health status indicators are illustrated for example in FIGS. 3A-3B, in other examples various other tracked health status indicators can be used, including more or less tracked health status indicators. Additionally, the values corresponding to the various tracked indicators can be derived from sensor data provided by sensors 112 or user input, or both.

FIG. 3C is a pictorial illustration showing one example display 502 that can be provided by health analysis system 122. As illustrated in FIG. 3C display 502 can include current symptoms display element 504, existing health conditions display element 506, predispositions display element 508, and action outcome display element 510. Current symptoms 504 indicates symptoms currently being experienced by the individual. Current symptoms 504 can be based on a variety of data, such as user input (e.g., self-reporting) or can be a predictive output generated by predictive analyzer component 146. Existing health conditions 506 indicates preexisting health conditions of the individual. Existing health conditions 506 can be based on a variety of data, such as user input and/or medical data 162. Predispositions 508 indicates health conditions to which the individual is predisposed (e.g., family history, genetic, given other conditions, medication side-effects, etc.). Predispositions 508 can be based on a variety of data, such as user input and/or medical data 162. Action outcome 510 indicates action(s) that were taken in response to the information indicated by 504, 506, and 508. In the illustrated example, a coronary angiogram was scheduled for the individual. In some examples, health analysis system 122 can generate an action signal to generate an indication, such as a recommendation to perform a certain medical service (e.g., coronary angiogram) based on the data. In some examples, health analysis system 122 can generate an action signal to schedule a certain medical service (e.g., coronary angiogram) based on the data. These actions signals can include causing interaction, such as by controlling communication system 128, with a remote system (e.g., health care provider computing system 116) and/or a remote user (e.g., healthcare worker 106).

The present discussion has mentioned processors and servers. In one embodiment, the processors and servers include computer processors with associated memory and timing circuitry, not separately shown. They are functional parts of the systems or devices to which they belong and are activated by, and facilitate the functionality of the other components or items in those systems.

Also, a number of user interface displays have been discussed. They can take a wide variety of different forms and can have a wide variety of different user actuatable input mechanisms disposed thereon. For instance, the user actuatable input mechanisms can be text boxes, check boxes, icons, links, drop-down menus, search boxes, etc. They can also be actuated in a wide variety of different ways. For instance, they can be actuated using a point and click device (such as a track ball or mouse). They can be actuated using hardware buttons, switches, a joystick or keyboard, thumb switches or thumb pads, etc. They can also be actuated using a virtual keyboard or other virtual actuators. In addition, where the screen on which they are displayed is a touch sensitive screen, they can be actuated using touch gestures. Also, where the device that displays them has speech recognition components, they can be actuated using speech commands.

A number of data stores have also been discussed. It will be noted they can each be broken into multiple data stores. All can be local to the systems accessing them, all can be remote, or some can be local while others are remote. All of these configurations are contemplated herein.

Also, the figures show a number of blocks with functionality ascribed to each block. It will be noted that fewer blocks can be used so the functionality is performed by fewer components. Also, more blocks can be used with the functionality distributed among more components.

It will be noted that the above discussion has described a variety of different systems, components and/or logic. It will be appreciated that such systems, components and/or logic can be comprised of hardware items (such as processors and associated memory, or other processing components, some of which are described below) that perform the functions associated with those systems, components and/or logic. In addition, the systems, components and/or logic can be comprised of software that is loaded into a memory and is subsequently executed by a processor or server, or other computing component, as described below. The systems, components and/or logic can also be comprised of different combinations of hardware, software, firmware, etc., some examples of which are described below. These are only some examples of different structures that can be used to form the systems, components and/or logic described above. Other structures can be used as well.

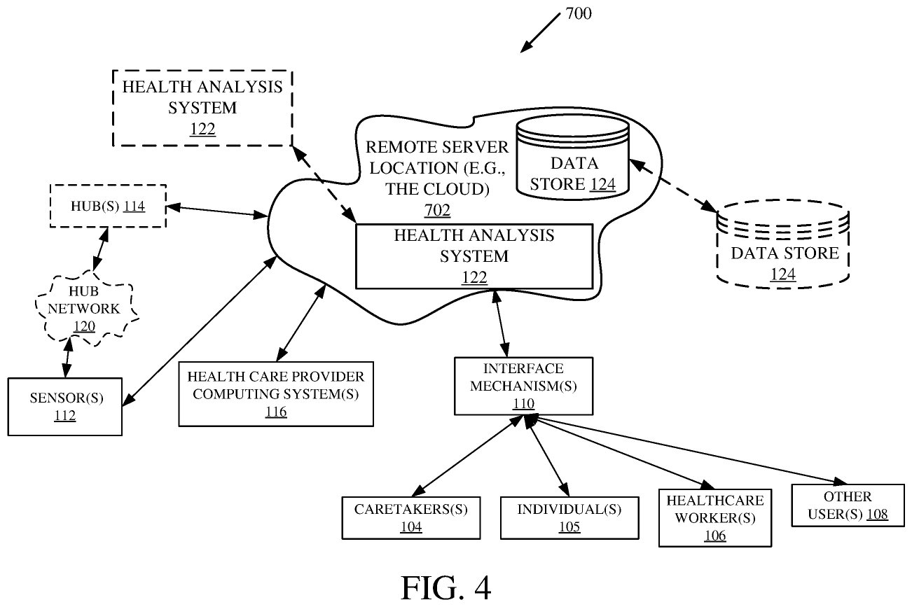

FIG. 4 is a block diagram of a remote server architecture, which shows that components of computing architecture 100 can communicate with elements in a remote server architecture, or that components of computing architecture 100 can be located at a remote server location and can be accessed at the remote server location by other components of computing architecture 100. In an example embodiment, remote server architecture 700 can provide computation, software, data access, and storage services that do not require end-user knowledge of the physical location or configuration of the system that delivers the services. In various embodiments, remote servers can deliver the services over a wide area network, such as the internet, using appropriate protocols. For instance, remote servers can deliver applications over a wide area network and they can be accessed through a web browser or any other computing component. Software or components shown in FIG. 1 as well as the corresponding data, can be stored on servers at a remote location. The computing resources in a remote server environment can be consolidated at a remote data center location or they can be dispersed. Remote server infrastructures can deliver services through shared data centers, even though they appear as a single point of access for the user. Thus, the components and functions described herein can be provided from a remote server at a remote location using a remote server architecture. Alternatively, they can be provided from a conventional server, or they can be installed on client devices directly, or in other ways.

In the embodiment shown in FIG. 4, some items are similar to those shown in FIG. and they are similarly numbered. FIG. 4 specifically shows that health analysis system 122 and data store 124 can be located at a remote server location 702. Therefore, users, such as caretaker(s) 104, individual(s) 105, healthcare worker(s) 106, and various other user(s) 108, and other items, such as health care provider computing system 116, sensors 112, and hubs 114, access those systems through remote server location 702.

FIG. 4 also depicts another embodiment of a remote server architecture. FIG. 4 shows that it is also contemplated that some elements of FIG. 1 are disposed at remote server location 702 while others are not. By way of example, health analysis system 122 or data store 124 can be disposed at a location separate from location 702, and accessed through the remote server at location 702. Regardless of where they are located, they can be accessed directly users, such as caretaker(s) 104, individual(s) 105, healthcare worker(s) 106, and various other user(s) 108, and other items, such as health care provider computing system 116, sensors 112, and hubs 114, through a network (either a wide area network or a local area network), they can be hosted at a remote site by a service, or they can be provided as a service, or accessed by a connection service that resides in a remote location. Also, the data can be stored in substantially any location and intermittently accessed by, or forwarded to, interested parties.

It will also be noted that the elements of FIG. 1 or portions of them, can be disposed on a wide variety of different devices. Some of those devices include servers, desktop computers, laptop computers, tablet computers, or other mobile devices, such as palm top computers, cell phones, smart phones, multimedia players, personal digital assistants, etc.

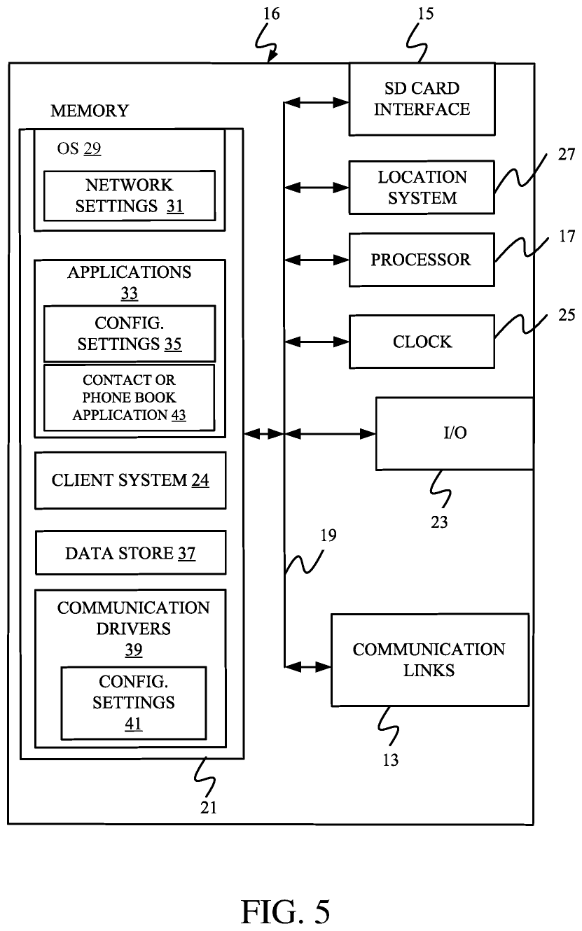

FIG. 5 is a simplified block diagram of one illustrative embodiment of a handheld or mobile computing device that can be used as a user's or client's hand held device 16, in which the present system (or parts of it) can be deployed. For instance, a mobile device can be used for generating, processing, or displaying the health levels, health analysis outputs, as well as various other information. FIGS. 5-7 are examples of handheld or mobile devices.

FIG. 5 provides a general block diagram of the components of a client device 16 that can run some components shown in FIG. 1, that interacts with them, or both. In the device 16, a communications link 13 is provided that allows the handheld device to communicate with other computing devices and under some embodiments provides a channel for receiving information automatically, such as by scanning. Examples of communications link 13 include allowing communication though one or more communication protocols, such as wireless services used to provide cellular access to a network, as well as protocols that provide local wireless connections to networks.

Under other embodiments, applications can be received on a removable Secure Digital (SD) card that is connected to an interface 15. Interface 15 and communication links 13 communicate with a processor 17 (which can also embody processor(s) 126 from FIG. 1) along a bus 19 that is also connected to memory 21 and input/output (I/O) components 23, as well as clock and location system 27.

I/O components 23, in one embodiment, are provided to facilitate input and output operations. I/O components 23 for various embodiments of the device 16 can include input components such as buttons, touch sensors, optical sensors, microphones, touch screens, proximity sensors, accelerometers, orientation sensors and output components such as a display device, a speaker, and or a printer port. Other I/O components 23 can be used as well.

Clock 25 illustratively comprises a real time clock component that outputs a time and date. It can also, illustratively, provide timing functions for processor 17.

Location system 27 illustratively includes a component that outputs a current geographical location of device 16. This can include, for instance, a global positioning system (GPS) receiver, a LORAN system, a dead reckoning system, a cellular triangulation system, or other positioning system. It can also include, for example, mapping software or navigation software that generates desired maps, navigation routes and other geographic functions.

Memory 21 stores operating system 29, network settings 31, applications 33, application configuration settings 35, data store 37, communication drivers 39, and communication configuration settings 41. Memory 21 can include all types of tangible volatile and non-volatile computer-readable memory devices. It can also include computer storage media (described below). Memory 21 stores computer readable instructions that, when executed by processor 17, cause the processor to perform computer-implemented steps or functions according to the instructions. Processor 17 can be activated by other components to facilitate their functionality as well.

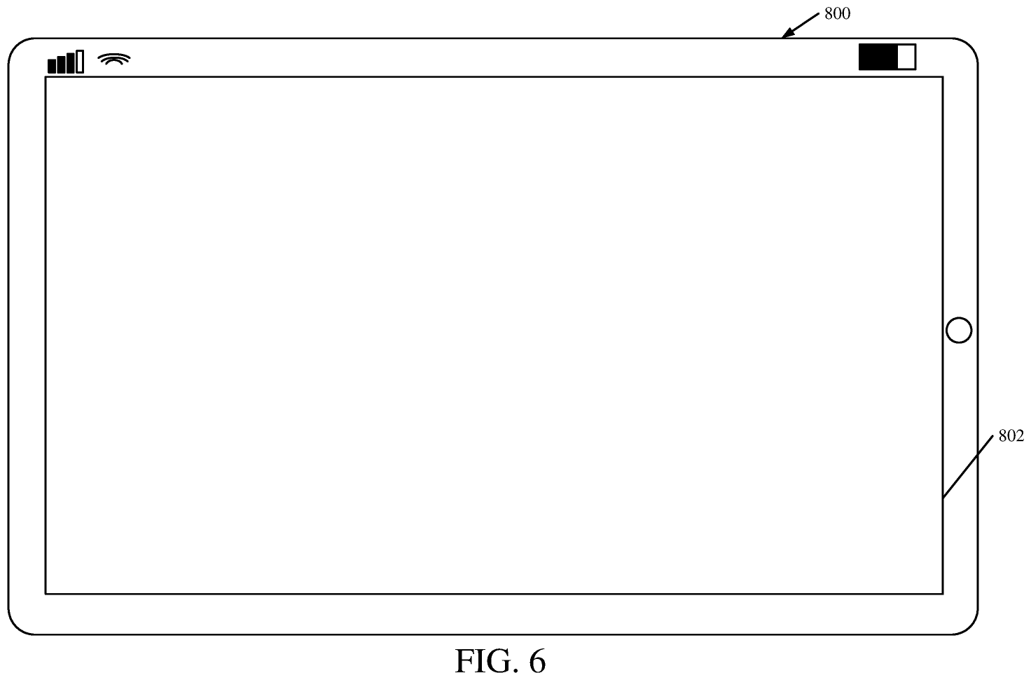

FIG. 6 shows one embodiment in which device 16 is a tablet computer 800. In FIG. 6, computer 800 is shown with user interface display screen 802. Screen 802 can be a touch screen or a pen-enabled interface that receives inputs from a pen or stylus. It can also use an on-screen virtual keyboard. Of course, it might also be attached to a keyboard or other user input device through a suitable attachment mechanism, such as a wireless link or USB port, for instance. Computer 800 can also illustratively receive voice inputs as well.

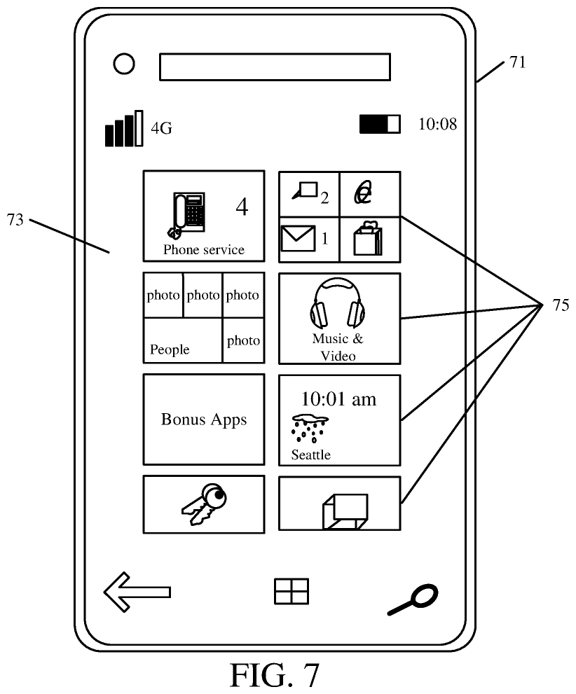

FIG. 7 is similar to FIG. 6 except that the device is a smart phone 71. Smart phone 71 has a touch sensitive display 73 that displays icons or tiles or other user input mechanisms 75. Mechanisms 75 can be used by a user to run applications, make calls, perform data transfer operations, etc. In general, smart phone 71 is built on a mobile operating system and offers more advanced computing capability and connectivity than a feature phone.

Note that other forms of the devices 16 are possible.

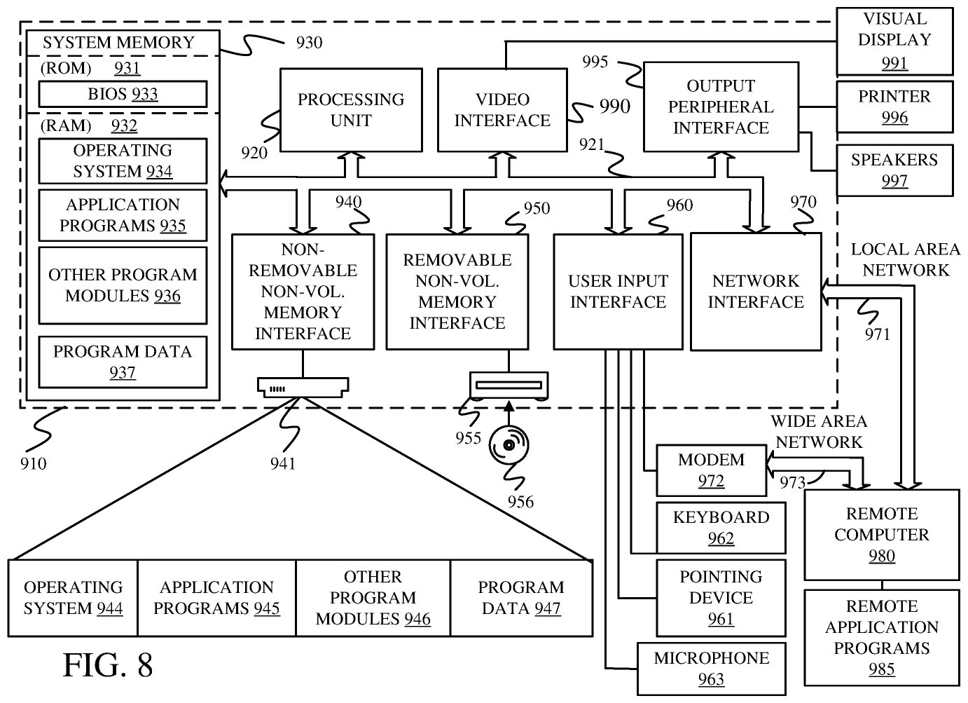

FIG. 8 is one embodiment of a computing environment in which elements of FIG. 1, or parts of it, (for example) can be deployed. With reference to FIG. 8, an exemplary system for implementing some embodiments includes a general-purpose computing device in the form of a computer 910. Components of computer 910 may include, but are not limited to, a processing unit 920 (which can embody processor(s) 126 of FIG. 1), a system memory 930, and a system bus 921 that couples various system components including the system memory to the processing unit 920. The system bus 921 may be any of several types of bus structures including a memory bus or memory controller, a peripheral bus, and a local bus using any of a variety of bus architectures. Memory and programs described with respect to FIG. 1 can be deployed in corresponding portions of FIG. 8.

Computer 910 typically includes a variety of computer readable media. Computer readable media can be any available media that can be accessed by computer 910 and includes both volatile and nonvolatile media, removable and non-removable media. By way of example, and not limitation, computer readable media may comprise computer storage media and communication media. Computer storage media is different from, and does not include, a modulated data signal or carrier wave. It includes hardware storage media including both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 910. Communication media may embody computer readable instructions, data structures, program modules or other data in a transport mechanism and includes any information delivery media. The term “modulated data signal” means a signal that has one or more of its characteristics set or changed in such a manner as to encode information in the signal.

The system memory 930 includes computer storage media in the form of volatile and/or nonvolatile memory such as read only memory (ROM) 931 and random access memory (RAM) 932. A basic input/output system 933 (BIOS), containing the basic routines that help to transfer information between elements within computer 910, such as during start-up, is typically stored in ROM 931. RAM 932 typically contains data and/or program modules that are immediately accessible to and/or presently being operated on by processing unit 920. By way of example, and not limitation, FIG. 8 illustrates operating system 934, application programs 935, other program modules 936, and program data 937.

The computer 910 may also include other removable/non-removable volatile/nonvolatile computer storage media. By way of example only, FIG. 8 illustrates a hard disk drive 941 that reads from or writes to non-removable, nonvolatile magnetic media, a magnetic disk drive 951, nonvolatile magnetic disk 952, an optical disk drive 955, and nonvolatile optical disk 956. The hard disk drive 941 is typically connected to the system bus 921 through a non-removable memory interface such as interface 940, and magnetic disk drive 951 and optical disk drive 955 are typically connected to the system bus 921 by a removable memory interface, such as interface 950.

Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (e.g., ASICs), Application-specific Standard Products (e.g., ASSPs), System-on-a-chip systems (SOCs), Complex Programmable Logic Devices (CPLDs), etc.

The drives and their associated computer storage media discussed above and illustrated in FIG. 8, provide storage of computer readable instructions, data structures, program modules and other data for the computer 910. In FIG. 8, for example, hard disk drive 941 is illustrated as storing operating system 944, application programs 945, other program modules 946, and program data 947. Note that these components can either be the same as or different from operating system 934, application programs 935, other program modules 936, and program data 937.

A user may enter commands and information into the computer 910 through input devices such as a keyboard 962, a microphone 963, and a pointing device 961, such as a mouse, trackball or touch pad. Other input devices (not shown) may include a joystick, game pad, satellite dish, scanner, or the like. These and other input devices are often connected to the processing unit 920 through a user input interface 960 that is coupled to the system bus, but may be connected by other interface and bus structures. A visual display 991 or other type of display device is also connected to the system bus 921 via an interface, such as a video interface 990. In addition to the monitor, computers may also include other peripheral output devices such as speakers 997 and printer 996, which may be connected through an output peripheral interface 995.

The computer 910 is operated in a networked environment using logical connections (such as a local area network—LAN, or wide area network WAN) to one or more remote computers, such as a remote computer 980.

When used in a LAN networking environment, the computer 910 is connected to the LAN 971 through a network interface or adapter 970. When used in a WAN networking environment, the computer 910 typically includes a modem 972 or other means for establishing communications over the WAN 973, such as the Internet. In a networked environment, program modules may be stored in a remote memory storage device. FIG. 8 illustrates, for example, that remote application programs 985 can reside on remote computer 980.

Further, example implementations of the invention(s) described herein may use one or more processors. If the implementation comprises multiple processors, they may be local or remote or a mixture, share information via wired, wireless, or utilizes a mixture of communication techniques, and/or fixedly or dynamically assign portions of computation to processors.

Processors may carry out their tasks with varying degrees of human supervision or intervention. Humans may be located at any appropriate process or communications node of the distributed system. Example human interaction devices without limitation include screens, touch screens, wearable displays, audio or speech output such as ear buds or speakers, microphones, haptic output such as vibration or thermal devices, brain wave sensors, eye trackers, heart rate and other physiological sensors, or cameras for facial, gesture, or other body monitoring.

In some examples, processors can include systems-on-a-chip, embedded processors, servers, desktop computers, tablet computer, or cell phones.

In some embodiments, unauthorized monitoring, altering, or substitution of data communications are mitigated. Without limitation, example embodiments may partially or fully implement authentication of nodes sending or receiving data, wherein the authentication techniques may include, without limitation, physical unclonable functions (PUFs), encryption of data sent between nodes, and/or use of a distributed, immutable ledger of data updates (e.g., Blockchain), as well as various other authentication techniques, or combinations thereof.

Some particular examples of the systems and methods described herein are provided below.

In one example, the systems and methods described herein proactively determines a lack of mobility that could lead to a fall, for instance, by using a mobility metric (e.g., score) that indicates how mobile an individual is. For example, upon detecting various activity changes, such as the individual not making it to the shower as often, not making it to the refrigerator as often, making fewer trips to the grocery store, the mobility metric may decrease, and the system or user can take action, such as to schedule physical therapy. Additionally, the system may recommend adding safety features to the home (e.g., handles, assist bars, ramps, etc.).

In one example, the systems and methods described herein determines when medication is taken each day and determines nonadherence to set schedules. The system can provide reminders, such as notifications or alerts on a device of the user, voice activated reminders, as well as reminder calls. Additionally, in some examples, the system can integrate medication management tracking with other activity tracking, such as leaving the stove on, driving out of range, etc., to proactively identify cognitive issues (e.g., memory loss, dementia, etc.).

In one example, systems and methods obtains a goal that is customizable by the individual. The system will calculate the variances against the goal so action can be taken such as more physical therapy or home health care to help with certain activities. Each displayed metric can contain a trend arrow showing if the metric is getting better or worse over time against the baseline and/or the goal that it is being compared to.

In one example, the systems and methods provides the family, caretaker, or patient with objective health and other activity metrics that assist the individual and their family in advocating for additional testing because they have multiple data points about their activities that show a significant physical, cognitive, or mental health decline. In addition, the systems and methods provides tracking and trending to indicated if certain health issues are a recurring problem to justify additional medical testing to search for the root cause.

In one example, the systems and methods assess potential issues with ADLs, IADLs, MHAs, and biomarkers, and uses them to calculate mobility, mental health, and cognitive scores. The system and methods can display these scores along with the individual's current or experienced symptoms, the individual's predispositions (e.g., heart disease, diabetes, etc.), and medical conditions that they currently are being treated for and/or diagnosed with (e.g., high blood pressure, etc.). The scores can be used in conjunction with detected biomarkers (e.g., blood pressure, EKG, blood glucose, etc.) to provide a more complete picture for medical professionals. The system can link all of these data points together and alert the individual if the data points relate back to the medical condition the individual is being treated for and/or is diagnosed with and/or the individual's predispositions. For example, if the patient has predispositions for heart disease and stroke and is or has experienced symptoms such as shortness of breath and confusion, and the activity variances show poor cognitive scores (e.g., indicated confusion), and low mobility scores then the system will alert the caretakers, health care workers, and/or health care providers that the individual should be seen as soon as possible. The system can communicate an increased severity level if more relationships exist to their predispositions and medical conditions being monitored.

In one example, the systems and methods provide for selection of and connectivity with devices, such as select sensors, to add those sensors to the computing architecture for interaction and sharing of information.

In one example, the systems and methods provide for calculating a baseline for each activity. The baseline can be calculated using data collected for a customizable timeframe. For example, a timeframe of 30 days may be used to collect data for a baseline. An average of the activity level over the timeframe can be used as the baseline, with the option to remove outliers. The system can also refresh the baseline with more current activity data if the individual or other users choose.

In one example, the systems and methods calculate the variance between detected activity and the baseline for that activity and/or the goal for that activity. In one example, the variance is calculated as the difference between the detected activity subtracted from the goal or baseline of that particular activity.

In one example, the systems and methods allow the individual or other users to provide input data indicative of the medical conditions the individual suffers from and/or are being treated for, and based on this input data, the system can alert the individual and/or other users about key symptoms to be on the lookout for. The system can alert and display this information with other key activities and metrics to give the individual and/or other users a customized and holistic picture of the individual's health.

In one example, the systems and methods provide messaging, such as video messaging, audio messaging, and/or text messaging between users of the system. In one example, the systems and methods provide for live video meeting between users of the system. In another example, the systems and methods provide for sharing data collected and generated by the system with others such as by presenting using online meeting, emailing, texting, or sharing a link to the information.

In one example, the systems and method provide for the individual and/or others to provide input data indicative of symptoms that are being experienced by the individual. The system can identify key words or key symptoms the individual is experiencing and determine if they are related to the health conditions that the individual is being monitored for and/or to the predispositions of the individual in the system, and/or to activity values and variances (e.g., eating, using the restroom, etc.), and/or to activity scores (e.g., mobility score, cognitive score, mental health score), and/or to biomarkers (e.g., high blood pressure, blood glucose, EKG readings, etc.). The system can alert the individual and/or other users if at least one piece of information (e.g., symptom) is related to the health condition being monitored, and/or to the predispositions, and/or to the activity variances, and/or to the activity scores, and/or to biomarkers. For example, if any symptoms being experienced were also related to the health condition being monitored, and/or to the predispositions then the system will alert the individual and/or other users that the symptoms the individual is experiencing in conjunction with the activity values and variances and/or activity scores, and/or biomarkers are all related, and that the individual should consult a medical professional as soon as possible. In one example, the systems and method provide for indicating varying severity in its alerts based on how many relationships are identified between symptoms the individual is experience and other health information (e.g., predispositions, health conditions, activity variances, activity scores, biomarkers, etc.). In one example, the systems and methods can provide for increased severity when reoccurring problems are identified.

In one example, the systems and methods can integrate information from an individual's health care provider. For example, the system will can receive appointment dates, symptoms reported at the appointment, and outcome from the appointment (e.g., medication prescribed, care prescribed, etc.). The system can accept, as an input, key actions that the medical professionals ask the individual to take (e.g., walk once per day, rest more often, get more sleep, schedule a follow-up appointment, etc.) and provide tracking related to these key actions.

In one example, the systems and methods can calculate the number of times an event has happened and identify the event as recurring based on how many other instances in the past the individual has experienced the event. The event can include symptoms, activity values and variances, activity scores, and/or biomarkers.

In one example, the systems and methods allow the individual or other users to provide input data that a doctor's appointment was scheduled. The system will also allow for the configuration of automatically accepting a new appointment date from the health care provider system when appointments are scheduled.

In one example, the systems and methods allow the individual or other users to provide input data indicative of an outcome from any medical professional visit. The outcome of the appointment may be a medication prescription, additional testing to be performed, or any other next steps from the medical professional. For example, if a medical visit happened, or medication was prescribed for a specific reason, then this can be entered into the system so that a complete record can be assessed across all data points (e.g., symptoms, predispositions, current health conditions being treated, changes in activities of daily living (ADLs, IADLs, etc.), changes in mental health activities, changes in biomarkers). This can be used by the system and/or the individual an/or other users to determine if the medicine and/or treatment prescribed by the medical professional results in improvement (e.g., improved symptoms, improved activity scores, improved biomarkers, etc.).

In one example, the systems and methods can use artificial intelligence (AI) and machine learning to predict the likelihood of an outcome based on the inputs being tracked by the system such as symptoms experienced, predispositions, current health conditions, activity values and variances, activity scores, and/or biomarkers. In one example, the system uses a function, such as a statistical regression, to predict the outcome using the inputs being tracked. The system can learn from new inputs and update the function on a real time basis. The system can also allow expert input to adjust the prediction function, such as to improve the accuracy of the prediction function.

In one example, the systems and methods allow healthcare workers to provide input data indicative of data from an appointment with the individual. For example, if a physical therapist has an appointment with the individual, the physical therapist can update the system with data of the appointment. The system will be able to show insights to the individual and/or other users if the physical therapy is increasing the activity scores (e.g., mobility scores) or not, and allow for the individual and/or other users (e.g., health care worker) to take other action to increase mobility of the patient.

In one example, the systems and methods can calculate a score (e.g., mobility score) that determines indicates whether an individual is getting more or less mobile over time. The system will track a combination of activities related to the individual's mobility (e.g., ADLs) to determine whether the individual's mobility is better or worse than the baseline or goal that the system is comparing against. The combination of activities that are used to determine mobility can be customized. In some examples, when at least one activity falls outside the baseline or goal by a calculated amount, then the system provides an indication of this variance, such as providing a display or other indication with different coloring to show a potential issue exists. An example of a group of activities that could be used to calculate the mobility score are the number of showers/baths the individual had during a week, the number of times the individual went up and down steps in the home, the number of times the individual went shopping (e.g., grocery shopping), and the number of times the individual traveled to a place outside the home (e.g., a community center).

In one example, the systems and methods can calculate a score (e.g., cognitive score) that indicates whether an individual cognitive health (e.g., memory) is getting better or worse over time. The system can use a combination of activities related to the individual's cognitive health (e.g., IADLs) to determine whether it is better or worse than the baseline or goal that the system is comparing against. The combination of activities that are used to determine cognitive health can be customized. In some examples, when at least one activity falls outside the baseline or goal by a calculated amount, then the system provides an indication of this variance, such as providing a display or other indication with different coloring to show a potential issue exists. An example of a group of activities that could be used to calculate the cognitive health score are taking medication on time, driving outside of a geographic limit, and forgetting to turn the stove off.

In one example, the systems and methods can calculate a score (e.g., mental health score) that indicates whether a client's mental health is getting better or worse over time. The system can use a combination of activities related to the individual's mental health (e.g., MHAs) to determine whether the individual's mental health is better or worse than the baseline or goal that the system is comparing against. The combination of activities that are used to determine mental health can be customized. In some examples, when at least one activity falls outside the baseline or goal by a calculated amount, then the system provides an indication of this variance, such as providing a display or other indication with different coloring to show a potential issue exists. An example of a group of activities that could be used to calculate the mental health score are going to a weekly spiritual service (e.g., church service, etc.), going to a social activity (e.g., daily coffee with friends at a coffee shop or other meeting location, going to bridge club, etc.), going to volunteer service (e.g., community service at a soup kitchen, etc.)

In one example, each score (e.g., mobility score, cognitive score, mental health score, biomarker score, etc.) can start with a score of 10. The system will subtract two points for each activity corresponding to the score that is more than 1 standard deviation from the baseline or goal, and 4 points for each activity that is more than 2 standard deviations from the baseline or goal. The score as displayed will turn a different color to alert the individual and/or other users when at least 1 activity corresponding to the score is worse than expected (e.g., baseline) or desired (e.g., goal). The score will turn a different color when at least 2 activities corresponding to the score are worse than expected (e.g., baseline) or desired (e.g., goal) to indicate more severity than when just at least 1 activity is worse. The system can also be customized to send an alert to the individual and/or other users when the score decreases or turn a different color, such as from at least 1 corresponding activity being more than 1 standard deviation from a baseline or a goal for that activity.

In one example, the systems and methods provide for alerting and messaging. The systems and methods allow the individual to transfer data collected and/or generated by the system to a healthcare worker (e.g., the individual's doctor) or health care provider system (e.g., the individual's hospital or clinic). This can include emailing, texting, or sharing a link with the healthcare worker or healthcare provider system. In one example, the systems and methods. In one example, the systems and methods allow the individual and/or other users define how they want to be communicated to by the system. For example, the individual and/or other users can select to receive email or text alerts based on specific criteria (e.g., activity scores or variances are worsening, activity score is decreasing, biomarkers are worsening, etc.), the individual and/or other users can select to receive an automated telephone call with an alert message based on specific criteria, and/or the individuals and/or other users can select for the system to provide displays that indicate an alert based on specific criteria (e.g., changing color to signify issue) or the individual and/or other users can use any combination of these alerting mechanisms.

In one example, the systems and methods provide for automatic or semi-automatic (e.g., user input) doctor appointment scheduling, such as when concerning trends are identified by the system (e.g., activity scores, values, or variances are worsening). The system will also allow the individual and/or other users to choose criteria (e.g., based on ADL levels, based on IADL levels, based on MHA levels, based on biomarker levels, etc.) for when an automatic doctor appointment gets scheduled, along with an alert to the individual and/or other users to indicate that an appointment has been scheduled. This scheduling functionality can be activated or deactivated by the individual and/or other users.

In one example, the systems and methods provide for customizable real time alerts based on data tracked by the system. For example, a real time alert when no motion is detected in the home for more than a threshold amount of time during a designated time of the day (e.g., no motion in the home for more than 1 hour during the afternoon). In another example, a real time alert when the system identifies that a door (e.g., front door) is left open longer than a threshold amount of time (e.g., longer than left open historically). In another example, a real time alert when the system determines a likely fall. In another example, a real time alert when the system identifies that the individual is not out of bed by a certain time. In another example, a real time alert when the system identifies that the individual has not taken medication by a certain time or has not taken medication during a given period of time (e.g., has not taken medication that day). In another example, a real time alert when the system identifies that the temperature of the home is unusually hot or cold. In another example, a real time alert when the system identifies that the individual was motionless in a certain room of the house for an unusually long period of time.

In one example, the cognitive score, mobility score, mental health score, and biomarker score, and symptoms that the individual is experience can be used by the system to predict outcomes and can also be used to provide alerts or other indications that individual is experiencing at least one indicator that individual could be experiencing a condition to which they are predisposed. Health and medical conditions have associated symptoms and/or indicators that are recognized by the health and medical community. The system can obtain (e.g., input into system, system accesses from third-party sources such as web-accessible websites) and store these associated symptoms and/or indicators, such as in a lookup (e.g., lookup table). For each predisposition of the individual (e.g., heart disease, stroke, etc.), the system can compare symptoms, biomarkers, activity data that are experienced by the individual. The system can determine if the experienced symptoms match the associated symptoms and/or indicators of predispositions of the individual, the system can provide an indication (e.g., alert) or other action signal. In another example, each score (e.g., cognitive score, mobility score, mental health score, biomarker score, etc.) can be correlated or otherwise tied, by the system, to different individual or different combinations of the associated symptoms and/or indicators. For example, the mobility score can be tied to stroke and/or heart disease, etc.; the mental health score can be tied to depression, anxiety, etc.; the biomarker score can be related to various predispositions (e.g., where the biomarker score is based on blood pressure it can be tied to heart disease or stroke, or both, etc.). The system can store these relationships (e.g., correlations/ties between scores and associated symptoms and/or indicators). In one example, where a score reduces lower than expected (e.g., lower than baseline/historical and/or goal and/or other threshold amount) and that score is related to (e.g., correlated/tied to) one or more predispositions of the patient, then the system can provide an alert (or other indication) or various other action signals.

It should also be noted that the different embodiments described herein can be combined in different ways. That is, parts of one or more embodiments can be combined with parts of one or more other embodiments. All of this is contemplated herein.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A computing system comprising:
a communication system configured to obtain sensor data indicative of performance, by an individual, of activity indicative of a health status of the individual;
a health analysis system configured to:
generate a value indicative of the performance of the activity indicative of the health status of the individual based on the sensor data; and
compare the value of the performance of the activity indicative of the health status of the individual to a reference value of performance of the activity indicative of the health status of the individual; and
an action signal generator configured to generate an action signal based on the comparison.

2. The computing system of claim 1, wherein the activity indicative of a health status of the individual comprises one or more activities of daily living (ADLs).

3. The computing system of claim 2, wherein the activity indicative of a health status of the individual further comprises one or more instrumental activities of daily living (IADLs).

4. The computing system of claim 1, wherein the activity indicative of a health status of the individual further comprises one or more mental health activities (MHAs).

5. The computing system of claim 4, wherein the one or more MHAs are selected from the group consisting of:
social activities;
spiritual activities; activities; and
volunteer activities.

6. The computing system of claim 4, wherein the health analysis system is further configured to:
generate ADL values indicative of a performance, by the individual, of one of the one or more ADLs;
generate IADL values indicative of performance, by the individual, of one of the one or more IADLs; and
generate NINA values indicative of performance, by the individual, of one of the one or more MHAs.

7. The computing system of claim 6, wherein the health analysis system is further configured to:
generate a mobility score, indicative of a mobility of the individual, based on a comparison of the generated ADL values to reference ADL values indicative of reference performance of the one or more ADLs.

8. The computing system of claim 6, wherein the health analysis system is further configured to:
generate a cognitive score, indicative of a cognitive health of the individual, based on a comparison of the generated IADL, values to reference IADL values indicative of reference performance of the one or more IADLs.

9. The computing system of claim 6, wherein the health analysis system is further configured to:
generate a mental health score, indicative of a mental health of the individual, based on a comparison of the generated MHA values to reference MHA values indicative of reference performance of the one or more MHAs.

10. The computing system of claim 6, wherein the communication system is further configured to obtain sensor data indicative of biomarkers of the individual; and
wherein the health analysis system is further configured to:
generate biomarker values indicative of levels of biomarkers of the individual, based on the sensor data indicative of biomarkers of the individual; and
generate a biomarker score, indicative of a biomarker health of the individual, based on a comparison of the generated biomarker values to reference biomarker values indicative of reference levels of biomarkers of the individual.

11. The computing system of claim 1, wherein the communication system is further configured to obtain sensor data indicative of a fall; and
wherein the health analysis system is configured detect a fall of the individual based on the sensor data indicative of the fall.

12. The computing system of claim 11, wherein the health analysis system is configured to identify a likely injury of the individual based on sensor data indicative of performance, by the individual, of activity indicative of the health status of the individual after the detected fall.

13. The computing system of claim 1, wherein the communication system is further configured to obtain:
data indicative of predispositions of the individual;
data indicative of symptoms associated with the predispositions; and
data indicative of symptoms currently being experienced by the individual; and
wherein the health analysis system is configured to determine that the individual is likely suffering from one or more of the predispositions, based on the data indicative of the predispositions of the individual, the data indicative of symptoms associated with the predispositions, and the data indicative of symptoms currently being experienced by the individual; and
wherein the action signal generator is configured to generate an action signal to communicate an alert to a user based on the determination that the individual is likely suffering from one or more of the predispositions.

14. The computing system of claim 12, wherein the health analysis system is configured to identify relationships between activity indicative of the health status of the individual and the predispositions of the individual and determine that the individual is likely suffering from one or more of the predispositions based on the comparison of the value of the performance of the activity indicative of the health status of the individual to the reference value of performance of the activity indicative of the health status of the individual.

15. The computing system of claim 1, wherein the reference value is based on historical values of performance, by the individual, of the activity indicative of the health status of the individual over a given period of time.

16. The computing system of claim 1, wherein the reference value is based on a user input indicative of a goal value of performance of the activity indicative of the health status of the individual.

17. The computing system of claim 1, wherein the action signal controls an interface mechanism to provide a display based on the comparison.

18. The computing system of claim 1, wherein the action signal automatically schedules an appointment with a health care provider based on the comparison.

19. The computing system of claim 1, wherein the action signal transmits an alert to a user based on the comparison.

20. A computer implemented method comprising:
obtaining sensor data indicative of performance, by an individual, of activity indicative of a health status of the individual;
generating a value indicative of the performance of the activity indicative of the health status of the individual based on the sensor data;
comparing the value of the performance of the activity indicative of the health status of the individual to a reference value of performance of the activity indicative of the health status of the individual; and
generating an action signal to provide an action based on the comparison.

21. The method of claim 20, wherein obtaining the sensor data comprises:
obtaining sensor data indicative of performance, by the individual, of one or more activities of daily living (ADLs);
obtaining sensor data, indicative of performance, by the individual, of one or more instrumental activities of daily living (IADLs); and
obtaining sensor data indicative of performance, by the individual, of one or more mental health activities (MHAs).

22. The method of claim 21, wherein generating the value indicative of the performance of the activity indicative of the health status of the individual comprises:
generating ADL values indicative of performance, by the individual, of the one or more ADLS;
generating IADL values indicative of performance, by the individual, of the one or more IADLs; and
generating MHA Values indicative of performance, by the individual, of the one or more MHAs.

23. The method of claim 22, wherein comparing the value of the performance of the activity indicative of the health status of the individual to a reference value of performance of the activity indicative of the health status of the individual comprises:
comparing the generated ADL values to reference ADL values indicative of reference performance of the one or more ADLs;
comparing the generated IADL values to reference IADL values indicative of reference performance of the one or more IADLs; and
comparing the generated MHA values to reference MHA Values indicative of reference performance of the one or more MHAs.

24. The method of claim 20, wherein comparing the value of the performance of the activity indicative of the health status of the individual to a reference value of performance of the activity indicative of the health status of the individual comprises:
comparing the value of the performance of the activity indicative of the health status of the individual to a historical value of performance of the activity indicative of the health status of the individual, the historical value indicative of a historical performance, by the individual, of the activity indicative of the health status of the individual.

25. The method of claim 20, wherein comparing the value of the performance of the activity indicative of the health status of the individual to a reference value of performance of the activity indicative of the health status of the individual comprises:
comparing the value of the performance of the activity indicative of the health status of the individual to a goal value of performance of the activity indicative of the health status of the individual, the goal value indicative of a goal performance, by the individual, of the activity indicative of the health status of the individual.

26. A computing system architecture comprising:
a sensor configured to detect performance, by an individual, of activity indicative of a health status of the individual and generate a sensor signal indicative of the detected performance;
a computing system comprising:
a communication system configured to obtain the sensor signal;
a health analysis system configured to:
generate a value indicative of the performance of the activity indicative of the health status of the individual based on the sensor signal; and
compare the value of the performance of the activity indicative of the health status of the individual to a reference value of performance of the activity indicative of the health status of the individual; and
an action signal generator configured to generate an action signal based on the comparison.

27. The computing system of claim 1, wherein the activity includes moving beyond a defined area.

28. The computing system of claim 1, wherein the activity includes leaving a residential door open within a defined period.

29. The computing system of claim 1, wherein the action signal generator provides a predictive action signal.
